(19) United States
(12) Patent Application Publication
(10) Pub. No.: US 2023/0310722 A1
(43) Pub. Date: Oct. 5, 2023

(54) PRESSURE SENSORS, INCLUDING OPTICAL PRESSURE SENSORS FOR AUTOMATED PERITONEAL DIALYSIS SYSTEMS, AND ASSOCIATED SYSTEMS, DEVICES, AND METHODS

(71) Applicant: BYONYKS MEDICAL DEVICES, INC., Itasca, IL (US)

(72) Inventors: Farrukh Usman, Chatham, NY (US); Michael Wollowitz, Chatham, NY (US); Naveed Iftikhar, Lahore (PK)

(21) Appl. No.: 18/041,623

(22) PCT Filed: Aug. 20, 2021
(86) PCT No.: PCT/US2021/047010

§ 371 Date: Feb. 14, 2023

Related U.S. Application Data
(60) Provisional application No. 63/068,384, filed on Aug. 21, 2020.

(51) Int. Cl. A61M 1/14

(52) U.S. Cl. CPC A61M 1/154; A61M 1/159; A61M 2205/3306; A61M 2205/3331

(57) ABSTRACT

Pressure sensors, including optical pressure sensors for automated peritoneal dialysis (APD) systems, and associated systems, devices, and methods are disclosed herein. In one embodiment, an APD system includes a diaphragm positioned over an opening in a cavity of a disposable set. The diaphragm has an outer surface and an inner surface opposite the outer surface. The diaphragm is configured to deform in response to a force applied against the diaphragm due to pressure of fluid within the cavity. The APD system further includes a pressure sensor configured to measure a pressure of the fluid within cavity. The pressure sensor includes a light source and a photosensor. The light source is configured to irradiate the outer surface of the diaphragm with light, and the photosensor is configured to measure an amount of the light that is reflected off of the outer surface of the diaphragm and directed

### CROSS-REFERENCE TO RELATED APPLICATION(S)

This application is a 371 U.S. National Phase of PCT/US2021/047010, filed Aug. 20, 2021, which claims the benefit of priority from U.S. Provisional Pat. Application No. 63/068,384, filed Aug. 21, 2020, both which are incorporated by reference herein in their entirety.

### TECHNICAL FIELD

The present disclosure is directed to pressure sensors, including optical pressure sensors for automated peritoneal dialysis (APD) systems, and associated systems, devices, and methods. For example, pressure sensors configured in accordance with some embodiments of the present technology are configured to measure pressure of isolated solutions flowing through disposable sets of APD systems.

### BACKGROUND

Dialysis is used to (a) remove excess fluid and toxins in persons with kidney failure and (b) correct electrolyte concentrations in their blood. Peritoneal dialysis is a form of dialysis that uses a peritoneum in an individual’s abdomen as a membrane through which fluid and dissolved substances are exchanged with blood. More specifically, a solution is introduced into and removed from the individual’s abdomen via a surgically installed catheter.

In continuous ambulatory dialysis (CAPD), solution is manually introduced and removed (e.g., at regular intervals throughout the day). In particular, the catheter is connected to a disposable set that includes (i) a source bag (e.g., hung on a drip stand) containing new solution, (ii) a drain bag configured to collect waste solution, and (iii) various fluid lines connecting the source bag and the drain bag to the catheter. Waste solution from the individual’s lower abdomen is drained into the drain bag via the catheter, and new solution is introduced into the individual’s lower abdomen via the catheter. After such an exchange treatment is complete, the disposable set is discarded.

APD (also known as continuous cycling peritoneal dialysis (CCPD)) is similar to CAPD except that the exchange treatment is automated using an APD machine or cycler. More specifically, a pump included in the APD machine is used to introduce and remove the solution (e.g., while the individual sleeps). Each APD exchange treatment may include one or more cycles of introducing and removing solution from the individual’s abdomen.

### BRIEF DESCRIPTION OF THE DRAWINGS

Many aspects of the present disclosure can be better understood with reference to the following drawings. The components in the drawings are not necessarily to scale. Instead, emphasis is placed on clearly illustrating the principles of the present disclosure. The drawings should not be taken to limit the disclosure to the specific embodiments depicted, but are for explanation and understanding only.

FIG. 1 is a partially schematic representation of an APD system configured in accordance with various embodiments of the present technology.

FIG. 2 is a partially schematic, cross-sectional, side view of a pressure sensor configured in accordance with various embodiments of the present technology.

FIGS. 3A-3D are partially schematic, cross-sectional, side views of a portion of a disposable set configured in accordance with various embodiments of the present technology.

FIGS. 4A-4C are partially schematic side views of a pressure sensor in operation in accordance with various embodiments of the present technology.

FIGS. 5A-5C are partially schematic side views of another pressure sensor in operation and configured in accordance with various embodiments of the present technology.

FIGS. 6 and 7 are partially schematic side views of still other pressure sensors in operation and configured in accordance with various embodiments of the present technology.

FIG. 8 is a flow diagram illustrating a method of measuring pressure of an isolated fluid in accordance with various embodiments of the present technology.

### DETAILED DESCRIPTION

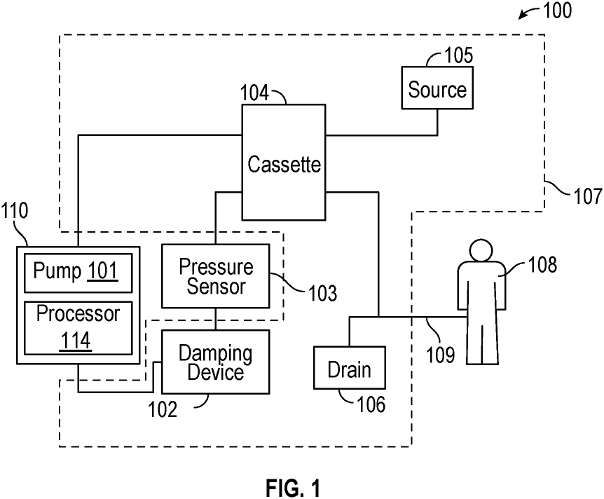

The present disclosure is directed to pressure sensors and associated systems, devices, and methods. In the illustrated embodiments below, pressure sensors of the present technology are primarily described in the context of optical pressure sensors measuring pressure of dialysate solution within or flowing through disposable sets of APD systems. Pressure sensors configured in accordance with various embodiments of the present technology, however, can be incorporated into and/or used by other systems, including hemodialysis systems and/or other medical or non-medical systems. Additionally, pressure sensors of the present technology can include non-optical pressure sensors and/or can be used to measure pressure of solutions or fluids besides dialysate solution, such as water, saline, blood, and/or other low viscous fluids. Furthermore, a person skilled in the art will understand that (i) the technology may have additional embodiments than illustrated in FIGS. 1-8 and (ii) the technology may be practiced without several of the details of the embodiments described below with reference to FIGS. 1-8.

### A. Overview

Many systems include pumps (e.g., peristaltic or other types of pumps in which fluid is isolated from pumping mechanisms) configured to regulate, control, and/or otherwise affect fluid flow through other components of the systems. For example, pumps are commonly used to perform blood transfusions and cardiopulmonary bypass operations. Pumps are also used in many industrial applications, such as agriculture or food dispensing. The act of pumping fluid creates fluid pressures within a system that can vary as the system is operated. In some systems, fluid pressures must remain within predetermined operating ranges to ensure safe or proper operation of the systems. For example, in some medical systems, when fluid pressure exceeds or violates safe operating limits, a patient may experience harm or discomfort. Thus, pressure sensors can be employed to monitor fluid pressure and ensure that the fluid pressures remain within safe operating ranges.

For certain systems (e.g., medical systems, laboratory systems, food dispensing systems, etc.), measuring and monitoring fluid pressure can be difficult because a fluid flowing through the systems must remain isolated to prevent contamination. In some of these systems, a disposable set of fluid lines and/or other components is used to convey fluid from a sterile container to a destination (e.g., a patient, a storage container, etc.). The disposable set can be pre-sterilized and disposed of after a single use to minimize the risk of contamination.

To measure fluid pressure in a disposable set, many systems use a pressure transducer that operates by converting (a) pressure of a fluid against a surface in contact with the fluid into (b) a displacement of some mechanical element (e.g., a force sensor) of the pressure transducer. Commonly, the displacement is then converted into an electrical signal that can be used to monitor the fluid pressure. For example, some industrial systems thread or press a pressure transducer into a port on a container or pipe. Such a solution has two primary drawbacks: (1) any port that opens into a sterile containment system is a potential source of contamination, and (2) the pressure transducer itself must be sterilized between uses and remains a potential source of contamination.

Another approach includes placing a thin, flexible membrane over an opening into a fluid containment system. The membrane has excess material so that it can deform with little resistance. A pressure transducer is centered on the membrane, and internal fluid pressure forces the membrane against a surface of the pressure transducer. A measured force can be used to approximate the internal pressure of fluid.

The above approach has previously been employed in hemodialysis systems. In a hemodialysis system, however, blood pressure ranges from about +6 kPa to about +30 kPa. Thus, the above approach is typically only used to measure positive fluid pressures. In addition, the membrane used in such a system is often fragile (e.g., easily deformed, extremely flexible, not rigid, not semi-rigid, etc.). Furthermore, the surface of the pressure transducer in contact with membrane is typically (a) planar and (b) much smaller than the opening in the disposable set transporting blood. When the planar surface contacts and/or deforms the membrane, the membrane is often stretched (e.g., at edges of the planar surface) and/or is not uniformly or smoothly deformed. This can lead to inaccuracies in pressure measurements captured by the pressure transducer and/or to inelastic deformation of the membrane. Furthermore, the planar surface does not fully support the membrane when the membrane is deformed, and the size of the planar surface (being much smaller than the opening) requires precise positioning of the surface at the center of the membrane for accurate measurement of blood pressure. As a result, such pressure transducers are often sensitive to variations in (i) thicknesses or moduli between different membranes of different disposable sets and/or (ii) different placements of the planar surface is relation to the center of the membrane. Therefore, complex analysis and experimentation is often required for each membrane and/or positioning of the planar surface to determine a relationship between a force measured by the pressure transducer and pressure of blood within the hemodialysis system.

Another approach that is used in applications in which high accuracy and measurement of low pressures are not needed involves measuring pressure in a soft elastomeric tube containing a pressurized fluid. The tube is partially flattened between two plates that are pressed against the tube from opposite sides. One of the plates is mounted on a force sensor. When fluid pressure within the tube works to restore a circular cross-section of the tube, the fluid pressure presses the tube against the plates and registers a force on the force sensor. The force can be used to approximate the pressure of the fluid within the tube.

In this approach, however, it is difficult (a) to maintain a consistent deformation of the tube and/or (b) to ensure that a force applied to restore the circular cross-section of the tube is fully transmitted to the force sensor. In addition, a large portion of a force measured by the force sensor is often due to a force provided by the tube itself to restore its circular cross-section rather than due to pressure of the fluid. Furthermore, this approach is often highly sensitive to variations in wall thickness, hardness, and/or other properties between different tubes. Moreover, it is difficult to measure negative pressures using this approach.

Other than pressure transducers, another approach used to measure fluid pressure in a disposable set involves including an electronic pressure sensor in the disposable set. In this approach, electrical leads of the sensor extend from an interior of the disposable set to contacts on an exterior of the disposable set. The disposable set (including the electronic pressure sensor) is sterilized before use and is then thrown away after use to eliminate the risk of contamination. Such an approach is therefore expensive and wasteful as the electronic pressure sensor is used only once.

One other approach is commonly used in APD systems in which dialysate is pumped into and out of a patient’s body. More specifically, APD systems typically employ systems that control fluid pressure by measuring air pressure external to the isolated, sterile dialysate. Such systems are often extremely expensive and complex.

To address the shortcomings of the approaches described above, the inventors have developed pressure sensors and associated systems, devices, and methods that are expected to safely, accurately, and affordably measure pressure of an isolated fluid (e.g., within or flowing through a disposable set). In one embodiment, a diaphragm is positioned over an opening of a cavity in a disposable set that includes one or more fluid lines. The diaphragm is affixed to the disposable set about a periphery of the opening. The APD system further includes a pressure sensor configured to measure a pressure of fluid flowing through the disposable set. The pressure sensor can include a light source and a photosensor. The light source can be configured to direct light at a reflector on an outer surface of the diaphragm. The reflector can reflect light toward the photosensor, and the photosensor can detect a portion of the reflected light.

When the diaphragm is deformed due to pressure of fluid flowing through the disposable set, the amount of reflected light that strikes and is detected by the photosensor changes. Thus, the photosensor can detect an amount of light reflected from the diaphragm, and the pressure sensor can use the amount of light detected to determine a pressure of the fluid flowing through the disposable set. In this manner, pressure sensors of the present technology can be used to monitor fluid pressures flowing through a disposable set and/or to increase the likelihood that the fluid pressures remain within safe operating ranges.

Pressure sensors and associated systems, devices, and methods of the present technology therefore offer several advantages. For example, the pressure sensor of the present technology indirectly contacts fluid via a diaphragm of a disposable set. Thus, the fluid can remain isolated within the disposable set (thereby reducing the risk of contaminating the fluid), and the pressure sensor can be repeatedly reused to measure pressure of fluid flowing through a plurality of different disposable sets (thereby reducing waste and costs of the system). Furthermore, the present technology can (a) compensate for effects due to temperature, aging, and/or variations in voltage and/or current supplied to the light source and/or the photosensor, and/or (b) factor out portions of a measurement captured by one or more photosensors that are not due to pressure of a fluid acting on the diaphragm. Thus, pressure sensors of the present technology can accurately determine pressure of the fluid flowing through the disposable set. Additionally, pressure sensors of the present technology can measure both positive and negative pressures in fluid flowing through a disposable set. As such, the present technology is particularly apt for APD and other systems that involve both infusion and aspiration of fluid. For example, the present technology can measure both a range of positive fluid pressures (e.g., 0 kPa to +10 kPa) that is commonly observed when introducing dialysate into a patient during a cycle of an APD treatment and a range of negative fluid pressures (e.g., 0 kPa to -10 kPa) that is commonly observed when removing dialysate from a patient during a cycle of an APD treatment.

### B. Selected Embodiments of Pressure Sensors, Including Pressure Sensors for APD Systems, and Associated Systems, Devices and Methods

FIG. 1 is a partially schematic representation of an APD system 100 (“the system 100”) configured in accordance with various embodiments of the present technology. As shown, the system 100 includes an APD machine 110 or cycler, a pressure sensor 103, and a disposable set 107. The disposable set 107 of FIG. 1 includes a damping device 102, a cassette 104, a source bag 105, a drain bag 106, and various fluid lines extending between components of the disposable set 107, the pressure sensor 103, and/or the APD machine 110. Other well-known components of APD systems are not illustrated in FIG. 1 or described in detail below so as to avoid unnecessarily obscuring aspects of the present technology.

In some embodiments, the APD machine 110 can include a pump 101. The pump 101 can be a non-invasive pump. For example, the pump 101 can be a peristaltic pump or another suitable type of pump. In these and other embodiments, the pump 101 and/or the pressure sensor 103 can be removably or permanently integrated into the APD machine 110. Alternatively, the pump 101 and/or the pressure sensor 103 can be components of the system 100 that are separate from the APD machine 110.

Various components of the disposable set 107 can interface with the APD machine 110. For example, the cassette 104 and/or the damping device 102 can be installed on (e.g., held in place, attached to, supported by, etc.) the APD machine 110 during an exchange treatment. As another example, a portion of the disposable set 107 (e.g., the cassette 104, the damping device 102, or another component of the disposable set 107) that includes a diaphragm or membrane (not shown) can be mounted or otherwise positioned on the APD machine 110 and/or aligned with the pressure sensor 103, as discussed in greater detail below. The disposable set 107 can be configured to interface (a) with the pump 101, (b) with the pressure sensor 103, and/or (c) with a catheter 109 installed in a patient 108. For example, the disposable set 107 can connect to the catheter 109 (e.g., directly or via a transfer set (not shown)) such that the catheter 109 is placed in fluid communication with the source bag 105 and/or the drain bag 106.

In operation, the system 100 can be configured to introduce solution (e.g., dialysate or another fluid initially contained within the source bag 105) into the patient 108 via the APD machine 110 (e.g., using the pump 101) and/or via at least a first portion of the disposable set 107. The system 100 can further be configured to remove solution from the patient 108 by draining the solution into the drain bag 106 (e.g., using the pump 101) and/or via at least a second portion of the disposable set 107. In some embodiments, a single exchange treatment can include one or more cycles of introducing solution into the patient 108 and removing solution from the patient 108. After an exchange treatment is complete, the disposable set 107 can be discarded and a separate (e.g., a new) disposable set 107 can be used for a subsequent treatment.

The cassette 104 is configured to control and/or direct solution flow through the disposable set 107. In some embodiments, the cassette 104 can be integrated with the damping device 102. The damping device 102 of the disposable set 107 can be configured to control, reduce, and/or minimize amplitudes of pressure pulsations in solution flowing through the damping device 102 and/or other components of the disposable set 107. For example, the damping device 102 can reduce amplitudes of positive and/or negative pressure pulsations that are induced in the solution by the pump 101 such that solution flow through the disposable set 107 is smoothed. This is expected to reduce, minimize, and/or eliminate patient discomfort while solution is pumped into and/or out of the patient 108. In these and other embodiments, the damping device 102 can be configured to remove air bubbles from solution within the damping device 102, which is expected to further reduce, minimize, and/or eliminate the possibility of patient discomfort or harm during an exchange treatment.

The pressure sensor 103 can be configured to measure pressure of solution within or flowing through at least a portion of the disposable set 107. For example, as discussed in greater detail below, a portion of the disposable set can be aligned with the pressure sensor 103, and the pressure sensor 103 can be configured to measure pressure of solution flowing through the portion of the disposable set 107 without the pressure sensor 103 coming in contact with the solution. The portion of the disposable set 107 aligned with the pressure sensor 103 can include a portion (e.g., a diaphragm or membrane) of the damping device 102, a portion (e.g., a diaphragm or membrane) of the cassette 104, or the portion of the disposable set 107 aligned with the pressure sensor 103 can be separate from the damping device 102 and the cassette 104.

As shown in FIG. 1, the system 100 and/or the APD machine 110 can include a processor 114. As discussed in greater detail below, the processor 114 and/or other components of the system 100 can monitor pressure measurements captured by the pressure sensor 103 and compare the pressure measurements to one or more safe operating thresholds or ranges. In these and other embodiments, the processor 114 and/or other components of the system 100 can interrupt an exchange treatment or cycle when one or more pressure measurements violate (e.g., exceed and/or drop below) the one or more safe operating thresholds or ranges.

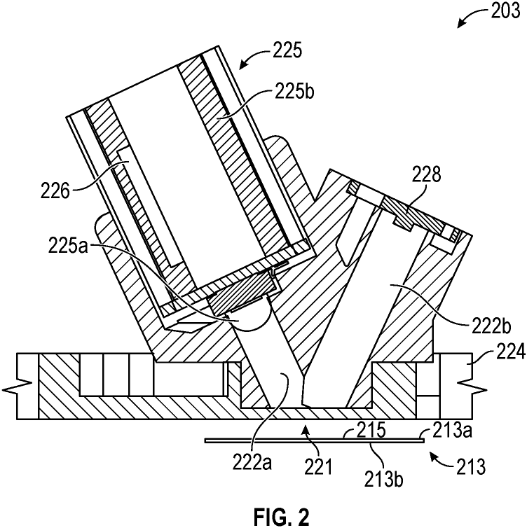

FIG. 2 is a partially schematic, cross-sectional, side view of a pressure sensor 203 configured in accordance with various embodiments of the present technology. The pressure sensor 203 can be the pressure sensor 103 of FIG. 1 or another pressure sensor configured in accordance with the present technology. As shown, the pressure sensor 203 includes a light source 225 and a photosensor 228. The light source 225 includes a light emitting element 225a (e.g., a bulb, a diode, etc.) and a heat sink 225b. In some embodiments, the light source 225 includes a light emitting diode (LED), a laser, or another compact light source. The light source 225 can be configured to project light having a wavelength inside the visible light spectrum or outside the visible light spectrum (e.g., corresponding to infrared or ultraviolet light). The photosensor 228 can include a photosensitive element or a photodetector.

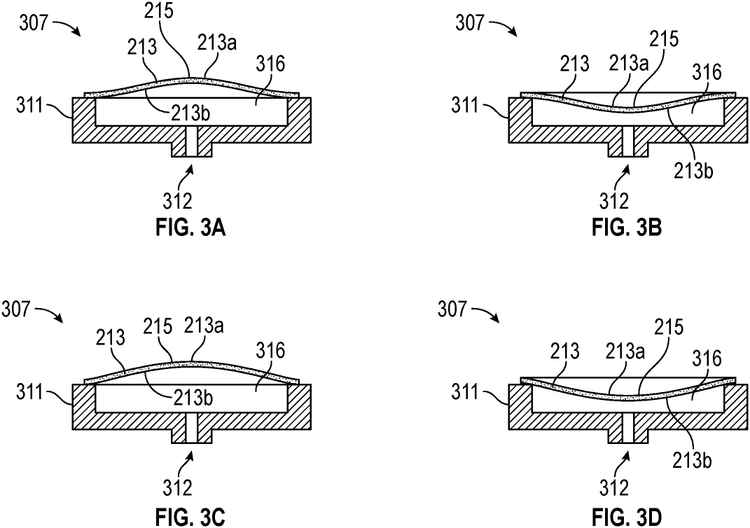

In operation, the light source 225 is configured to direct light (e.g., at an angle) toward a diaphragm 213 that is aligned with the pressure sensor 203. In turn, light emitted from the light source 225 can reflect off an outer surface 213a of the diaphragm 213 (e.g., at an angle) and be redirected toward the photosensor 228. In this regard, the light source 225 and the photosensor 228 can be positioned along a common optical axis that is folded at an intersection with the outer surface 213a of the diaphragm 213. As discussed in greater detail below with respect to FIG. 3A-7, an amount of light emitted from the light source 225 and detected by the photosensor 228 along the common optical axis can depend at least in part on a force acting against an inner surface 213b of the diaphragm 213 opposite the outer surface 213a due to pressure of a fluid or solution (e.g., within a disposable set). Thus, the amount of light detected by the photosensor 228 can be used to determine a pressure of the fluid acting upon the diaphragm 213.

In some embodiments, the pressure sensor 203 further includes a mount or housing 224 that can retain the light source 225 and/or the photosensor 228 in a desired position, orientation, and/or spacing. In these and other embodiments, the mount 224 can define at least a portion of a first light baffle 222a and/or at least a portion of a second light baffle 222b. Alternatively, the first light baffle 222a and/or the second light baffle 222b can be components of the system that are separate from the mount 224. The first light baffle 222a and/or the second light baffle 222b can be configured to shield (a) the photosensor 228 and/or light emitted from the light source 225 from (b) ambient light. Additionally, or alternatively, the first light baffle 222a can be configured to limit an area of light emitted from the light source 225 such that the light (a) exits an opening 221 in the mount 224 and/or in the first light baffle 222a and (b) primarily strikes a central area of the outer surface 213a of the diaphragm 213. In these and other embodiments, the second light baffle 222b can be configured to limit an area of light that (a) is reflected from the diaphragm 213, (b) reenters the opening 221 or another opening in the mount 224 and/or the second light baffle 222b, and/or (c) strikes the photosensor 228.

In some embodiments, the first light baffle 222a and/or the second light baffle 222b can include darkened (e.g., black) sidewalls. The darkened sidewalls can facilitate absorption of light that is emitted from the light source 225 off of the common optical axis and/or that is reflected from the diaphragm 213 off of the common optical axis. Stated another way, the darkened sidewalls of the first light baffle 222a and/or the second light baffle 222b can reduce, minimize, and/or eliminate the likelihood that light projected off of the common optical axis is reflected off of the sidewalls of the first light baffle 222a and/or the second light baffle 222b and/or is detected by the photosensor 228. In these and other embodiments, the first light baffle 222a and/or the second light baffle 222b can include lightened (e.g., white) or reflective sidewalls. The lightened or reflective sidewalls can facilitate reflection of light that is emitted from the light source 225 off of the common optical axis and/or that is reflected from the diaphragm 213 off of the common optical axis. Stated another way, the lightened or reflective sidewalls of the first light baffle 222a and/or the second light baffle 222b can increase, maximize, and enhance the likelihood that light projected off of the common optical axis is reflected off of the sidewalls of the first light baffle 222a, is reflected off of the diaphragm 213, is reflected off of the sidewalls of the second light baffle 222b, and/or is detected by the photosensor 228. As a specific example, the first light baffle 222a can include lightened or reflective sidewalls (e.g., to increase the amount of light that reaches and/or is reflected off of the diaphragm 213), and the second light baffle 222b can include darkened sidewalls (e.g., to reduce the likelihood that light reflected off of the diaphragm 213 off of the common optical axis strikes and/or is detected by the photosensor 228). In other embodiments, the first light baffle 222a and/or the second light baffle 222b can be omitted, and light can intrinsically be directed from the light source 225 to the diaphragm 213 and/or from the diaphragm 213 to the photosensor 228 along the common optical axis. In some embodiments, the light source 225 and/or the light baffles 222 can be selected to reduce or enhance an effect in which an intensity of light emitted from the light source 225 is at a maximum along its central axis and gradually decreases away from that axis.

To obtain accurate pressure measurements, the pressure sensor 203 can include one or more electronic circuits (not shown) that are configured to supply a constant current to the light source 225 (e.g., to maintain a stable light output over time) and/or a constant voltage to the photosensor 228 (e.g., to maintain a stable light sensitivity over time). Current and voltage can be dependent upon a number of factors, including temperature. Thus, the pressure sensor 203 can include one or more temperature sensors 226 in some embodiments to help compensate for fluctuations in temperature of various components of the pressure sensor 203 and/or for fluctuations in temperature of the ambient atmosphere surrounding components of the pressure sensor 203 over time. For example, in the embodiment illustrated in FIG. 2, the pressure sensor 203 includes a temperature sensor 226 positioned generally along the heat sink 225b of the light source 225. Additionally, or alternatively, the pressure sensor 203 can include a temperature sensor 226 positioned at other locations along the light source 225. In these and other embodiments, the pressure sensor 203 can include one or more temperature sensors 226 positioned at other locations. For example, the pressure sensor 203 can include a temperature sensor 226 positioned along the photosensor 228. In operation, the temperature sensor(s) 226 can be configured to capture one or more temperature measurements of various components (e.g., the light source 225, the photosensor 228, etc.) of the pressure sensor 203. The temperature measurements can be communicated to the electronic circuits, and the electronic circuits can use the temperature measurements to reduce variations in the current or voltage supplied to the light source 225 and/or to the photosensor 228.

Referring now to the diaphragm 213, the diaphragm 213 can be aligned with the pressure sensor 203 (e.g., using the mount 224 or another mount or holder of the pressure sensor 203 of the APD system). For example, the diaphragm 213 can be positioned, centered, and/or held beneath the opening 221 and/or another opening of the mount 224. In these and other embodiments, the diaphragm 213 can be positioned, centered, and/or held along the common optical axis described above. In these and still other embodiment, the diaphragm 213 can be positioned, centered, and/or held at a fixed distance from the opening 221, the light source 225, and/or the photosensor 228.

As shown in FIG. 2, the diaphragm 213 has a thin, sheet-like structure. The diaphragm 213 can be opaque or transparent. In some embodiments, at least the outer surface 213a of the diaphragm 213 is smooth and/or has a relatively low coefficient of friction. The material(s) used to form the diaphragm 213, the thickness of the diaphragm 213, and/or the size/shape of the diaphragm 213 can depend on a range of pressures to be applied to the diaphragm 213, a type of fluid applying the pressure, and/or the required accuracy of pressure measurements calculated from measurements captured by the photosensor 228. For example, the diaphragm 213 can be made of metal, ceramic, glass, and/or polymer (e.g., polycarbonate, polyethylene terephthalate (PET), polymethylpentene, etc.) depending upon application. As another particular example, the diaphragm 213 can have (a) a diameter from about 15 mm to about 40 mm and/or (b) a thickness ranging from about 0.2 mm to about 0.5 mm. In some embodiments, the diaphragm 213 has a circular or disk shape (e.g., to match a circular or disk shape of an opening in a structure to which the diaphragm is attached). Other shapes (e.g., triangular, rectangular, pentagonal, etc.) and dimensions for the diaphragm 213 and/or the opening of the rim structure 211 are of course possible and within the scope of the present technology.

In some embodiments, the outer surface 213a of the diaphragm 213 can include a specular (e.g., mirror-like) reflector 215. For example, the outer surface 213a can include a polished or plated metal or metallic coating disposed on a smooth polymer substrate. The metallic coating can be aluminized PET (also known as reflective Mylar) or another metallic coating. As another example, the outer surface 213a can include a thin, flexible reflective foil or aluminum tape applied to a thicker and/or stiffer diaphragm substrate. In some embodiments, the reflector 215 can cover all of the outer surface 213a of the diaphragm 213. In other embodiments, the reflector 215 can cover a subset (e.g., a majority, just a central area, etc.) of the outer surface 213a of the diaphragm 213.

The diaphragm 213 can be attached, affixed, and/or integrated with a portion of a disposable set, such as the disposable set 107 of FIG. 1. For example, FIGS. 3A-3D are partially schematic, cross-sectional, side views of a portion 307 of a disposable set (e.g., the disposable set 107 of FIG. 1) that includes the diaphragm 213 of FIG. 2 and that is configured in accordance with various embodiments of the present technology. In addition to the diaphragm 213, the portion 307 of the disposable set can include a rim structure 311 and a port 312 (e.g., a tube, a channel, etc.). In some embodiments, the diaphragm 213 is affixed (e.g., hermetically and/or using an adhesive) to the rim structure 311 at a periphery of an opening at a top portion of the rim structure 311. In other embodiments, the diaphragm 213 can be integrated with the rim structure 311. For example, the diaphragm 213 and the rim structure 311 can be molded as a single component, with a portion of the single component corresponding to the diaphragm 213 being thinner and more flexible than a portion of the single component corresponding to the rim structure 311.

The rim structure 311 and/or the diaphragm 213 define (at least in part) a cavity 316. The cavity 316 can be rigid (e.g., at least along the portions corresponding to the rim structure 311). Additionally, or alternatively, the cavity 316 can be closed (e.g., hermetically sealed) by the rim structure 311 and/or the diaphragm 213 except for the port 312. The port 312 can fluidly connect the cavity 316 to fluid lines (not shown) or other portions of the disposable set. In these embodiments, as fluid flows through the disposable set, the fluid can enter the cavity 316 via the port 312 and exert a force on the diaphragm 213. Although shown within only one port 312 in FIG. 2, the portion 307 of the disposable set can include a greater number of ports 312 (e.g., an inlet port 312 and an outlet port 312) in other embodiments of the present technology.

The diaphragm 213 can be a rigid, semi-rigid, or semi-flexible structure. In some embodiments, a positive internal pressure within the cavity 316 (relative to external pressure) will cause the diaphragm 213 to deform outwardly while a negative internal pressure within the cavity 316 (relative to external pressure) will cause the diaphragm 213 to deform inwardly. FIGS. 3A-3D, for example, illustrate various possible deformations of the diaphragm 213. The amount of deformation illustrated in FIGS. 3A-3D is greatly exaggerated for the sake of clarity and understanding. In actuality, the amount of deformation is often not readily perceivable.

In FIGS. 3A and 3B, the diaphragm 213 and the rim structure 311 include a clamp-type joint at locations where the diaphragm 213 is affixed to the rim structure 311. Thus, the diaphragm 213 deforms primarily by bending. More specifically, a central part of the diaphragm 213 deforms with a curvature opposite that of an outer part of the diaphragm 213. For positive pressure within the cavity 316 (FIG. 3A), the central part of the diaphragm 213 bends outwardly such that it is convex (viewed from outside the cavity 316) while the outer part of the diaphragm 213 is concave. For negative pressure within the cavity 316 (FIG. 3B), the central part of the diaphragm 213 bends inwardly such that it is concave (viewed from outside the cavity 316) while the outer part of the diaphragm 213 is convex.

In FIGS. 3C and 3D, the diaphragm 213 and the rim structure 311 include a flexible-type joint at locations where the diaphragm 213 is affixed to the rim structure 311. Thus, the diaphragm 213 deforms primarily by stretching. More specifically, most or all of the diaphragm 213 deforms with a same curvature. For positive pressure within the cavity 316 (FIG. 3C), both the central part and the outer part of the diaphragm 213 bend outwardly such that they are convex (viewed from outside the cavity 316). For negative pressure within the cavity 316 (FIG. 3D), both the central part and the outer part of the diaphragm 213 bend inwardly such that they are concave (viewed from outside the cavity 316). This type of deformation of the diaphragm 213 often occurs when the maximum displacement (e.g., at the center) of the diaphragm 213) is somewhat greater than the thickness of the diaphragm 213.

As discussed above, fluid enters the cavity 316 of the portion 207 of the disposable set via the port 312 and exerts a force against the diaphragm 213. The force is related to a pressure of the fluid within the cavity 316. Thus, when fluid pressure is positive, the fluid exerts a force that causes the diaphragm 213 to deform outwardly. When fluid pressure is negative, the fluid exerts a force that causes the diaphragm 213 to deform inwardly. As the diaphragm 213 deforms inwardly or outwardly, an amount of light (a) emitted from the light source 225 (FIG. 2) of the pressure sensor 203 (FIG. 2) and (b) reflected off of the outer surface 213a (e.g., off of the specular reflector 215) of the diaphragm 213 along the common optical axis and toward the photosensor 228 (FIG. 2) changes. Thus, the pressure sensor 203 can measure an amount of light detected by the photosensor 228 and use the measurements to determine a pressure of the fluid within the cavity 316 without contacting with the fluid. Therefore, when the fluid is dialysate or another medical solution flowing through a disposable set (e.g., for introduction into a patient), the fluid can remain isolated (thereby reducing the risk of contaminating the fluid before introduction of the fluid into the patient), and the pressure sensor 203 can calculate a pressure of the fluid flowing through the disposable set. In turn, pressure measurements captured by the pressure sensor 203 can be monitored to ensure that the fluid pressure remains within safe ranges, thereby reducing, minimizing, and/or eliminating the risk of patient harm or discomfort from pressure of the fluid as it is introduced into the patient.

In some embodiments, the portion 307 of the disposable set can be a portion of a damping device (e.g., the damping device 102 of FIG. 1) or a portion of a cassette (e.g., the cassette 104 of FIG. 1). For example, a damping device can include one or more diaphragms having one or more cavities configured to reduce pressure pulsations in fluid flowing through the disposable set. Continuing with this example, a diaphragm, a body portion, and/or a cavity of the damping device can correspond to the diaphragm 213, the rim structure 311, and/or the cavity 316 of FIGS. 3A-3C. Thus, the pressure sensor 203 can be configured to determine a pressure of fluid flowing through the cavity of the damping device. Further details regarding damping devices can be found in International (PCT) Application No. PCT/US2021/027428, which is incorporated by reference herein in its entirety.

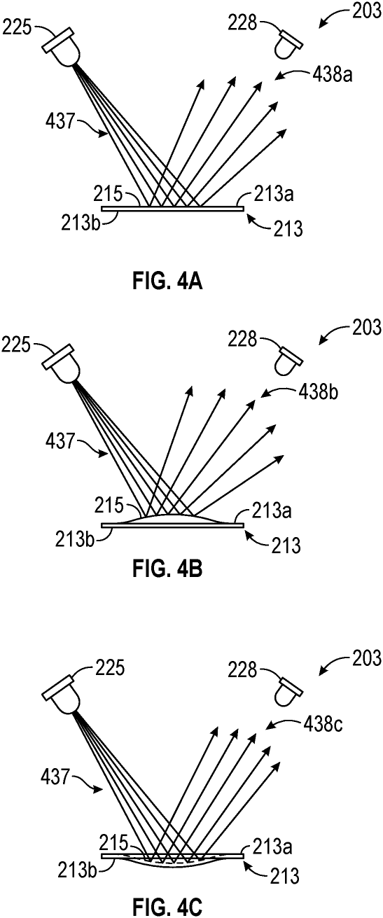

FIGS. 4A-4C are partially schematic side views of the pressure sensor 203 of FIG. 2 in operation in accordance with various embodiments of the present technology. Only the light source 225, the photosensor 228, and the diaphragm 213 are shown in FIGS. 4A-4C. The other components (e.g., the light baffles 222, the mount 224, the rim structure 311, the port 312, the cavity 316, etc.) of FIG. 2-3C are not shown in FIGS. 4A-4C to avoid unnecessarily obscuring aspects of the present technology.

Referring first to FIG. 4A, the diaphragm 213 is flat because there is no pressure (other than atmospheric pressure) acting on the diaphragm 213. Light 437 is emitted from the light source 225 and is directed (e.g., at a first angle) toward the reflector 215 on the outer surface 213a of the diaphragm 213. As the light 437 strikes the reflector 215, at least a portion of the light 437 is reflected (e.g., at a second angle) generally toward the photosensor 228. A portion 438a of the light 437 that is reflected off of the diaphragm 213 strikes and is detected by the photosensor 228.

In some embodiments, the area of the light 437 is limited by intrinsic directionality of the light source 225 and/or by use of the first light baffle 222a (FIG. 2) such that the light 437 emitted from the light source 225 primarily strikes a central region of the outer surface 213a of the diaphragm 213 (e.g., a portion of the outer surface 213a of the diaphragm 213 corresponding to the reflector 215). Additionally, or alternatively, the light 437 emitted from the light source 225 can have an intensity that is at a maximum along its central axis and that gradually decreases away from the central axis. As discussed above, this effect can be reduced or enhanced via appropriate choice of a light source 225 and the first light baffle 222a.

As the light 437 is emitted from the light source 225, the light 437 begins to diverge. The light 437 continues to diverge after it is reflected off of the diaphragm 213, and some of the light 437 can be blocked, absorbed, and/or reflected by the first and/or second light baffles 222 (FIG. 2). As such, the portion 438a of the light 437 that strikes and is detected by the photosensor 228 typically represents a smaller amount of the light 437 than is originally emitted from the light source 225. In some embodiments, the amount of light represented by the portion 438a of the light 437 in FIG. 4A can be used as a baseline or zero-offset value for calculations of positive (FIG. 4B) and negative (FIG. 4C) pressure applied to the diaphragm 213 by fluid or solution within the cavity 316 (FIGS. 3A-3C).

FIG. 4B illustrates the diaphragm 213 under positive pressure. As shown, the diaphragm 213 forms a convex, diverging mirror under positive pressure. Thus, when light 437 is emitted from the light source 225 and strikes the reflector 215 on the outer surface 213a of the diaphragm 213, the convex form of the diaphragm 213 contributes to an increase in a divergence of the light 437 after the light 437 is reflected off of the diaphragm 213 in comparison to the divergence of the light 437 after reflection when the diaphragm is flat (FIG. 4A). The solid angle of the light 437 in FIG. 4B is increased (in comparison to the solid angle of the light 437 of FIG. 4A) while the solid angle of the photosensor 228 remains constant (in comparison to the solid angle of the photosensor 228 of FIG. 4A). In turn, a portion 438b of the light 437 that strikes and is detected by the photosensor 228 is smaller than the portion 438a of FIG. 4A. In other words, an amount of the light 437 that is detected by the photosensor 228 when the diaphragm 213 has a convex form as a result of positive pressure (FIG. 4B) is smaller than an amount of the light 437 that is detected by the photosensor 228 when the diaphragm 213 is flat in the absence of pressure (FIG. 4A).

FIG. 4C illustrates the diaphragm 213 under negative pressure. As shown, the diaphragm 213 forms a concave, converging mirror under negative pressure. Thus, when light 437 is emitted from the light source 225 and strikes the reflector 215 on the outer surface 213a of the diaphragm 213, the concave form of the diaphragm 213 contributes to a decrease in a divergence (or to an increase in convergence) of the light 437 after the light 437 is reflected off of the diaphragm 213 in comparison to the divergence of the light 437 of FIGS. 4A and 4B after reflection off of the diaphragm 213. The solid angle of the light 437 in FIG. 4C is decreased (in comparison to the solid angles of the light 437 of FIGS. 4A and 4B) while the solid angle of the photosensor 228 remains constant (in comparison to the solid angles of the photosensor 228 in FIGS. 4A and 4B). In turn, a portion 438c of the light 437 that strikes and is detected by the photosensor 228 is larger than the portions 438a and 438b of FIGS. 4A and 4B. In other words, an amount of the light 437 that is detected by the photosensor 228 when the diaphragm 213 has a concave form as a result of negative pressure (FIG. 4C) is larger than (a) an amount of the light 437 that is detected by the photosensor 228 when the diaphragm 213 is flat in the absence of pressure (FIG. 4A) and (b) an amount of the light 437 that is detected by the photosensor 228 when the diaphragm has a convex form as a result of positive pressure (FIG. 4B).

Referring now to FIGS. 4A-4C together, variations in the amount of the light 437 that is detected by the photosensor 228 can be converted by electronic elements (not shown) of the pressure sensor 203 into corresponding analog variations of voltage, current, frequency, or pulse width. The corresponding analog variations can be converted into corresponding digital outputs. The analog and/or digital values output by the electronic elements typically have a monotonic and/or non-linear relationship with pressure applied to the diaphragm 213. This relationship can be characterized using a variety of methods, including characterizing the relationship based on test data for a specific diaphragm 213, pressure range, and/or optical geometry. As discussed above with respect to FIG. 4A, calculations of pressure can be based at least in part on a baseline or zero-offset value representing an amount of light 437 that is captured by the pressure sensor 203 in the absence of pressure applied to the diaphragm 213 (e.g., after the diaphragm 213 is aligned with the pressure sensor 213 but before a pressure is applied to the diaphragm 213). As such, pressure sensors 203 configured in accordance with various embodiments of the present technology are able to determine a pressure of fluid within the cavity 316 (FIGS. 3A-3C) based at least in part on an amount of light 437 that is detected by the photosensor 228.

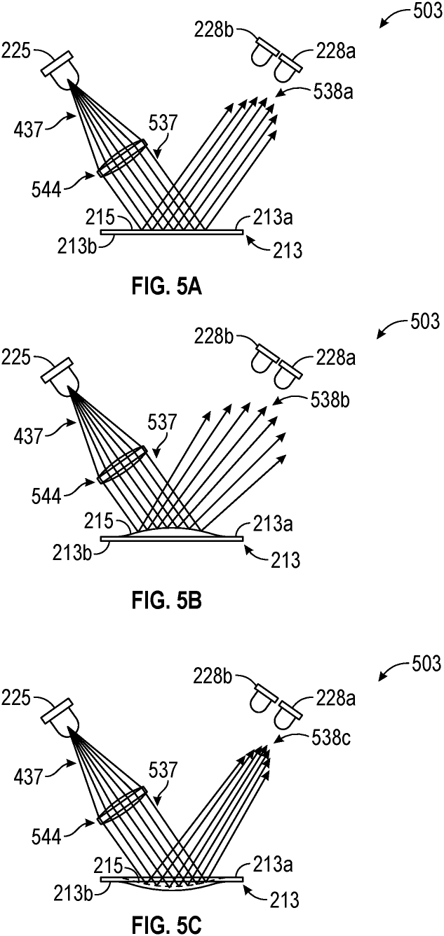

FIGS. 5A-5C are partially schematic side views of another pressure sensor 503 in operation and configured in accordance with various embodiments of the present technology. The pressure sensor 503 can be the pressure sensor 103 of FIG. 1 or another pressure sensor configured in accordance with the present technology. As shown, the pressure sensor 503 is generally similar to the pressure sensor of FIGS. 2 and 4A-4C except that the pressure sensor 503 can include a collimating element 544 and/or a plurality of photosensors 228 (identified individually as first photosensor 228a and second photosensor 228b in FIGS. 5A-5C).

The collimating element 544 of the pressure sensor 503 can be a lens, a concave mirror, or another optical element. The collimating element 544 is configured to convert light 437 that is emitted from the light source 225 into collimated light 537. More specifically, the collimating element 544 can be positioned within the first light baffle 222a (FIG. 2) and/or within a pathway of the light 437 emitted from the light source 225 (e.g., between the light source 225 and the diaphragm 213). As light 437 is emitted from the light source 225, the light 437 can be collimated by the collimating element 544 such that collimated light 537 that is non-diverging (or is slightly diverging or slightly converging) strikes the reflector 215 on the outer surface 213a of the diaphragm 213. In some embodiments, the collimating element 544 can have a size and/or geometry such that the collimated light 537 strikes all or a subset of the reflector 215 at a central area of the diaphragm 213. This can increase and/or maximize the effects of convexity or concavity of the diaphragm 213 on the divergence or convergence of the collimated light 537 as the collimated light 537 is reflected off of the diaphragm 213.

In these and other embodiments, the collimating element 544 and/or other components of the pressure sensor 503 and/or an APD system including the pressure sensor 503 can be configured (e.g., spaced, positioned, and/or oriented relative to one another) such that collimated light 537 that is reflected off of the diaphragm 213 and directed generally toward the photosensor(s) 228 can cover an area at the location of the photosensor(s) 228 that is larger (e.g., by a factor of 1.5, 2.0, or more) than a sensitive area of the photosensor(s) 228, even when the collimated light 537 is most concentrated (e.g., when diaphragm 213 is concave under negative pressures). Such an arrangement can prevent a loss of sensitivity to negative pressures as the diaphragm 213 forms a concave, converging mirror. Such an arrangement can additionally, or alternatively, decrease a sensitivity of the pressure sensor 503 to alignment of the light source 225, the diaphragm 213, and/or the photosensor(s) 228.

Referring to FIG. 5A, the diaphragm 213 is flat because there is no pressure (other than atmospheric pressure) acting on the diaphragm 213. As the collimated light 537 strikes the reflector 215 of the diaphragm 213, the collimated light 537 is reflected generally toward the photosensor(s) 228 and generally maintains its collimated structure. In turn, a portion 538a of the light 537 strikes and is detected by the photosensor(s) 228. The portion 538a of the collimated light 537 that strikes and is detected by the photosensor(s) 228 typically represents a smaller amount of the light 437 than is originally emitted from the light source 225. In some embodiments, the amount of light represented by the portion 538a of the collimated light 537 in FIG. 5A can be used as a baseline or zero-offset value for calculations of positive (FIG. 5B) and negative (FIG. 5C) pressures applied to the diaphragm 213 by fluid or solution within the cavity 316 (FIGS. 3A-3C).

FIG. 5B illustrates the diaphragm 213 forming a convex, diverging mirror under positive pressure, and FIG. 5C illustrates the diaphragm 213 forming a concave, converging mirror under negative pressure. The pressure sensor 503 under positive and negative pressures can operate generally similar to the pressure sensor 203 (FIGS. 4A-4C) under positive and negative pressures, respectively. For example, as the diaphragm 213 deforms into a convex mirror under positive pressure, divergence of the collimated light 537 after the collimated light 537 is reflected off of the diaphragm 213 increases. As a result, a portion 538b (FIG. 5B) of the collimated light 537 that strikes and is detected by the photosensor(s) 228 represents a smaller amount of the collimated light 537 than the amount of the collimated light 537 represented by the portion 538a of the collimated light 537 that strikes and is detected by the photosensor(s) 228 in FIG. 5A when no pressure is applied to the diaphragm 213. As another example, as the diaphragm 213 deforms into a convex mirror under negative pressure, divergence of the collimated light 537 after the collimated light 537 is reflected off of the diaphragm 213 decreases (or the convergence of the collimated light 537 after the collimated light 537 is reflected off of the diaphragm 213 increases). As a result, a portion 538c (FIG. 5C) of the collimated light 537 that strikes and is detected by the photosensor(s) 228 represents a larger amount of the collimated light 537 than the amounts of the collimated light 537 represented by (a) the portion 538a of the collimated light 537 that strikes and is detected by the photosensor(s) 228 in FIG. 5A when no pressure is applied to the diaphragm 213 and (b) the portion 538b of the collimated light 537 that strikes and is detected by the photosensor(s) 228 in FIG. 5B when a positive pressure is acting upon the diaphragm 213.

In some embodiments, the collimated light 537 reflected off of the diaphragm 213 and toward the photosensor(s) 228 under no pressure (FIG. 5A), positive pressures (FIG. 5B), and/or negative pressures (FIG. 5C) is typically more concentrated than the light 437 that is reflected off of the diaphragm 213 and toward the photosensor 228 under no pressure (FIG. 4A), positive pressures (FIG. 4B), and/or negative pressures (FIG. 4C), respectively. As such, the portions 538a, 538b, and/or 538c of the collimated light 537 that strike and are detected by the photosensor(s) 228 in FIGS. 5A, 5B, and/or 5C typically represent larger amounts of light than the amounts of light that are represented by the portions 438a, 438b, and/or 438c, respectively, of the light 437 that strike and are detected by the photosensor 228 in FIGS. 4A, 4B, and/or 4C. In turn, analog and/or digital outputs of electronic elements (not shown) of the pressure sensor 503 of FIGS. 5A-5C can be larger (e.g., have higher amplitudes or magnitudes) than respective analog and/or digital outputs of the electronic elements of the pressure sensor 203 of FIGS. 4A-4C.

In these and other embodiments, for a given range of pressures, variation in the amounts of the collimated light 537 that are detected by the photosensor(s) 228 in FIGS. 5A-5C (e.g., variation between the portions 538a, 538b, and 538 of FIGS. 5A-5C) can be greater than variation in the amounts of the light 437 that are detected by the photosensor 228 in FIGS. 4A-4C (e.g., variation between the portions 438a, 438b, and 438c). In turn, variations in the analog and/or digital outputs of the electronic elements of the pressure sensor 503 can be larger (e.g., have higher amplitudes or magnitudes) than respective variations in the analog and/or digital outputs of the electronic elements of the pressure sensor 203. The larger variations in the analog and/or digital outputs of the pressure sensor 503 can facilitate an increase in accuracy of calculations of pressure of fluid in the cavity 316 (FIGS. 3A-3D) and/or an increase in sensitivity to changes of pressure of the fluid in the cavity 316.

As discussed above, the pressure sensor 503 can include a plurality of photosensors 228. For example, the pressure sensor 503 of FIGS. 5A-5C includes a first photosensor 228a and a second photosensor 228b. In some embodiments, the first photosensor 228a can be similar or identical to the second photosensor 228b. In other embodiments, the first photosensor 228a can be different from the second photosensor 228b.

The first photosensor 228a can be positioned at a location generally similar to the photosensor 228 of FIGS. 4A-4C. The second photosensor 228b can be positioned adjacent to the first photosensor 228a and spaced apart from an axis between the diaphragm 213 and the first photosensor 228a along which collimated light 537 reflected from the diaphragm 213 generally travels toward the first photosensor 228a. In FIGS. 5A-5C, the second photosensor 228b is generally oriented parallel to the axis between the diaphragm 213 and the first photosensor 228a. In other embodiments, the second photosensor 228b can be generally oriented in a different direction, such as in a direction generally perpendicular to the axis between the diaphragm 213 and the first photosensor 228a. Regardless of the general orientation of the second photosensor 228b, the second photosensor 228b can include a light sensitive region that faces in a same general direction as a light sensitive region of the first photosensor 228a.

As shown in FIGS. 5A-5C, the light source 225 provides a certain amount of luminous flux, and the analog and/or digital outputs corresponding to the first photosensor 228a depend at least in part on a concentration of the luminous flux that reaches the light sensitive element of the photosensor 228a. Because variation in a concentration of a fixed amount of luminous flux at a first location (e.g., a location corresponding to the light sensitive element of the first photosensor 228a) can imply an opposite variation in a concentration of the fixed amount of luminous flux at a second location, the light sensitive element of the second photosensor 228b can be positioned at the second location such that the second photosensor 228b detects the opposite variation. In other words, the first photosensor 228a and the second photosensor 228b can be arranged relative to one another such that (a) the second photosensor 228b detects a decreasing concentration of luminous flux from the light source 225 as the first photosensor 228a detects an increasing concentration of the luminous flux from the light source 225, and/or (b) the second photosensor 228b detects an increasing concentration of luminous flux from the light source 225 as the first photosensor 228a detects a decreasing concentration of luminous flux from the light source 225. In some embodiments, the collimated light 537 (at least after being reflected off of the diaphragm 213) can have an intensity that gradually decreases with distance away from its central axis. This can ensure that intensity of the collimated light 537 at locations off of the central axis varies smoothly with (as opposed to being discontinuous with) intensity of the collimated light 537 on the central axis.

Referring to FIG. 5B as a specific example, when positive pressure is applied to the diaphragm 213 and the diaphragm 213 deforms into a convex, diverging mirror, the first photosensor 228a can detect lesser light intensities in comparison to the light intensity detected by the first photosensor 228a when no pressure is applied to the diaphragm 213 and the diaphragm 213 is flat (FIG. 5A). Thus, the output of the first photosensor 228a can be reduced. At the same time, the second photosensor 228b can detect greater light intensities in comparison to the light intensity detected by the second photosensor 228b when no pressure is applied to the diaphragm 213 and the diaphragm 213 is flat. Thus, the output of the second photosensor 228b can be increased.

Referring to FIG. 5C as an additional example, the opposite effects can be observed when negative pressure is applied to the diaphragm 213 and the diaphragm 213 deforms into a concave, converging mirror. In particular, the first photosensor 228a can detect greater light intensities in comparison to the light intensity detected by the first photosensor 228a when no pressure is applied to the diaphragm 213 and the diaphragm 213 is flat (FIG. 5A). Thus, the output of the first photosensor 228a can be increased. At the same time, the second photosensor 228b can detect lesser light intensities in comparison to the light intensity detected by the second photosensor 228b when no pressure is applied to the diaphragm 213 and the diaphragm 213 is flat. Thus, the output of the second photosensor 228b can be decreased.

The first photosensor 228a and the second photosensor 228b of the pressure sensor 503 can therefore be used to produce a differential output representing an amount of the collimated light 537 detected that varies with pressure applied to the diaphragm 213 by fluid in the cavity 316 (FIGS. 3A-3D). In some embodiments, the differential output can be used to (a) improve linearity in the relationship between pressure of a fluid in the cavity 316 and the amount of light detected by one or both of the photosensor(s) 228; (b) improve strength of the analog and/or digital outputs of the electronic elements of the pressure sensor 503; (c) reject or factor out components of the analog and/or digital outputs of the electronic elements of the pressure sensor 503 that are not due to pressure applied to the diaphragm 213 by fluid in the cavity 316; and/or (d) facilitate repeatable results of the pressure sensor 503.

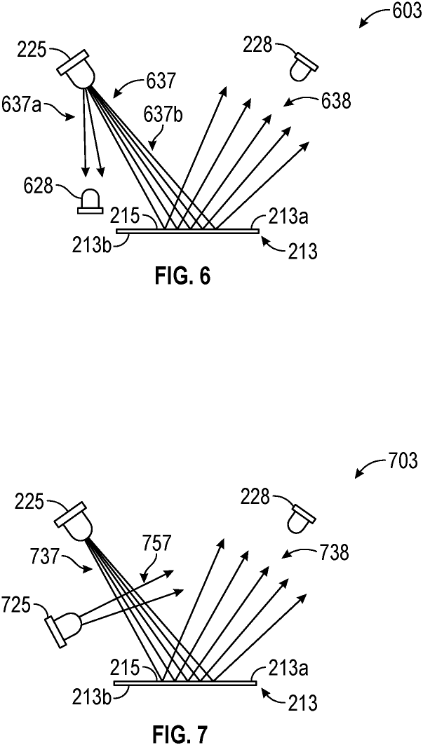

FIGS. 6 and 7 are partially schematic side views of still other pressure sensors 603 and 703, respectively, in operation and configured in accordance with various embodiments of the present technology. Referring to FIG. 6, the pressure sensor 603 can be generally similar to the pressure sensor 203 (FIGS. 2 and 4A-4C) and/or the pressure sensor 503 (FIGS. 5A-5C) except that the pressure sensor 603 can include a photosensor 628 having a light sensitive element directed generally toward the light source 225 (as opposed to the diaphragm 213). In some embodiments, the photosensor 628 can be similar or identical to the photosensor(s) 228. In other embodiments, the photosensor 628 can be different from the photosensor(s) 228.

In operation, the photosensor 628 is configured to receive a first portion 637a of light 637 emitted from the light source 225 that is not directed toward the diaphragm 213 while a second portion 637b of the light 637 is emitted from the light source 225 and directed toward the diaphragm 213. As such, the photosensor 628 can provide a measurement of the light 637 emitted from the light source 225 that is not dependent upon reflectance off of the diaphragm 213 (in contrast to the portion 638 of the light 637 detected by the photosensor(s) 228). The measurement captured by the photosensor 628 can be used to detect and/or compensate for variations in the light 637 emitted from the light source 225 that can occur due to variations in (a) voltage or current supplied to the light source 225, (b) a temperature of the light source 225, and/or (c) aging of the light source 225.

Referring to FIG. 7, the pressure sensor 703 can be generally similar to the pressure sensor 203 (FIGS. 2 and 4A-4C), the pressure sensor 503 (FIGS. 5A-5C), and/or the pressure sensor 603 (FIG. 6) except that the pressure sensor 703 can include a light source 725 configured to project light 757 directly onto the photosensitive element(s) of the photosensor(s) 228. In some embodiments, the light source 725 can be similar or identical to the light source 225. In other embodiments, the light source 725 can be different from the light source 225.

The light 757 projected onto the photosensor(s) 228 can provide a measure of sensitivity of the photosensor(s) 228 that is not dependent upon reflectance off of the diaphragm 213 (in contrast to the portion 738 of the light 737 emitted by the light source 225 and detected by the photosensor(s) 228). The measure of sensitivity of the photosensor(s) 228 that is provided by the light 757 from the light source 725 can be used to detect and/or compensate for variations in the sensitivity of the photosensor(s) 228 that can occur due to variations in (a) voltage or current supplied to the photosensor(s) 228; (b) a temperature of the photosensor(s) 228, and/or (c) aging of the photosensor(s) 228.

Although various components of pressure sensors of the present technology are illustrated and described above in separate embodiments, any combination of these components can be employed in pressure sensors configured in accordance with various other embodiments of the present technology. For example, a pressure sensor of the present technology can include the light source 225 (FIGS. 2 and 4A-7), the photosensor 228 (FIGS. 2 and 4A-4C), the first photosensor 228a (FIGS. 5A-5C), the second photosensor 228b (FIGS. 5A-5C), the collimating element 544 (FIGS. 5A-5C), the photosensor 628 (FIG. 6), and/or the light source 725 (FIG. 7). In embodiments having multiple photosensors and/or multiple light sources, the photosensors can be similar or identical to one another, and/or the light sources can be similar or identical to one another. In these and other embodiments, one or more of the photosensors can be as closely spaced as is practical and/or can be supplied power from a common power source. Additionally, or alternatively, one or more of the light sources can be as closely spaced as is practical and/or can be supplied power from a common power source. The similarity, close spacing, and/or common power sources between photosensors and/or between light sources can increase the likelihood that effects due to temperature and/or electrical power (e.g., supplied voltage and/or current) are common to all of them. In turn, this can result in more accurate and/or effective compensation for the effects due to temperature and/or electrical power. In other embodiments, photosensors can be different from one another, light sources can be different from one another, spacing between photosensors can be larger than in other embodiments of the present technology, spacing between light sources can be larger than in other embodiments of the present technology, photosensors can be supplied power from different power sources, and/or light sources can be supplied power from different power sources.

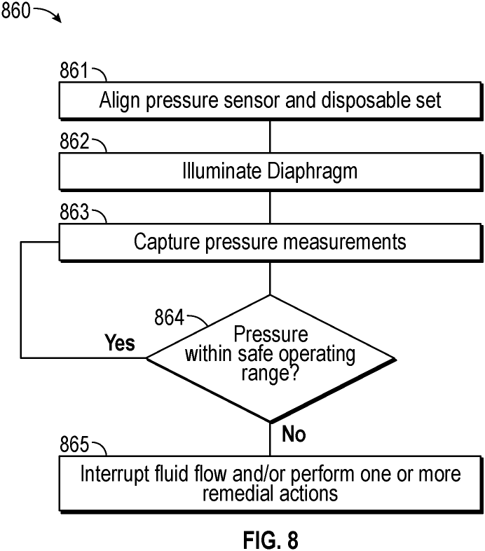

FIG. 8 is a flow diagram illustrating a method 860 of measuring pressure of an isolated fluid in accordance with various embodiments of the present technology. For example, the method 860 can be a method of measuring pressure of a fluid (e.g., a dialysate or another solution) within or flowing through a portion (e.g., the portion 307 of FIGS. 3A - 3D) of a disposable set (e.g., the disposable set 107 of FIG. 1) of an APD system (e.g., the system 100 of FIG. 1). The method 860 is illustrated as a set of blocks, steps, operations, or processes 861-865. All or a subset of the blocks 861-865 can be executed at least in part by various components of a system, such as the APD system 100 of FIG. 1. For example, all or a subset of the blocks 861-865 can be executed at least in part by a pump, a pressure sensor, a damping device, a cassette, fluid lines, and/or other portions of a disposable set. Additionally, or alternatively, all or a subset of the blocks 861-865 can be executed at least in part by an operator (e.g., a user, a patient, a caregiver, a family member, a physician, etc.) of the system. Furthermore, any one or more of the blocks 861-865 can be executed in accordance with the discussion above.

The method 860 begins at block 861 by aligning a disposable set with a pressure sensor. In some embodiments, aligning the disposable set with the pressure sensor can include aligning a diaphragm of portion of the disposable set with the pressure sensor. For example, FIG. 2 illustrates the diaphragm 213 aligned with the pressure sensor 203 such that the diaphragm 213 is positioned beneath an opening 221 in the first light baffle 222a and/or in the second light baffle 222b, is positioned in a pathway of light emitted from the light source 225, and/or is positioned such that a reflector 215 on an outer surface 213a of the diaphragm 213 can reflect light generally toward the photosensor 228. A mount or clamp (e.g., on an APD machine) can be employed to stably and removably position the portion of the disposable set in a fixed orientation and/or spacing relative to the pressure sensor as part of the alignment process. For example, an operator can position the portion of the disposable set into a mount. Additionally, or alternatively, the pressure sensor can be moveable such that it can be aligned with the portion of the disposable set. All or a subset of the block 861 of the method 860 can be performed without a fluid actively flowing through the disposable set and/or before a fluid enters a cavity of the portion (e.g., the cavity 316 of the portion 307 of FIGS. 3A-3D) of the disposable set.

At block 862, the method 860 continues by irradiating or illuminating the diaphragm. Irradiating the diaphragm can include emitting light from the light source and/or directing the light (e.g., at a first angle and/or along a first axis) toward an outer surface and/or a reflector of the diaphragm. Irradiating the diaphragm can include shielding the light emitted from the light source from ambient light using a first light baffle. In these and other embodiments, irradiating the diaphragm includes absorbing, using the first light baffle, portions of the light emitted from the light source that diverge from the first axis. In these and still other embodiments, irradiating the diaphragm includes limiting an area of the light emitted from the light source using the first light baffle such that a portions of the light that exits the opening 221 primarily strikes a central region of the diaphragm corresponding at least in part to the reflector. Additionally, or alternatively, irradiating the diaphragm includes collimating the light emitted from the light source using a collimating element such that the diaphragm is illuminated or irradiated with collimated light.

Irradiating the diaphragm can include irradiating the diaphragm when no pressure (e.g., other than atmospheric pressure) is applied to the diaphragm or when a positive or negative pressure is applied to the diaphragm. For example, the method 860 can irradiate the diaphragm when no pressure is applied to the diaphragm to determine a zero-offset value for pressure calculations. As another example, the method 860 can irradiate the diaphragm when positive and/or negative test pressures are applied to the diaphragm to determine a relationship between an amount of light detected by one or more photosensors of the pressure sensor and pressures applied to the diaphragm. As still another example, the method 860 can irradiate the diaphragm to measure pressure of fluid within the cavity of the portion of the disposable set.

At block 863, the method 860 continues by capturing one or more pressure measurements. In some embodiments, capturing a pressure measurement includes reflecting light emitted from the light source (e.g., at a second angle and/or along a second axis) toward one or more photosensors. Reflecting the light can include reflecting the light using a reflector and/or an outer surface of the diaphragm. Capturing a pressure measurement can include shielding light reflected from the diaphragm from ambient light using a second light baffle. In these and other embodiments, capturing a pressure measurement includes absorbing, using the second light baffle, portions of the light reflected from the diaphragm that diverge from the second axis. In these and still other embodiments, capturing a pressure measurement includes limiting an area of the reflected light that enters and/or travels along the second light baffle.

Additionally, or alternatively, capturing a pressure measurement includes capturing one or more amounts of the reflected light using one or more photosensors. For example, capturing a pressure measurement can include using a single or only one photosensor to detect an amount of light reflected from the diaphragm. As another example, capturing a pressure measurement can include using multiple photosensors, such as a first photosensor and a second photosensor, to detect multiple amounts of light reflected from the diaphragm and/or to determine a differential signal of the amount of light reflected from the diaphragm. In these and other embodiments, capturing a pressure measurement includes capturing an analog signal corresponding to an amount of light reflected from the diaphragm and/or detected by a photosensor. In these and still other embodiments, capturing a pressure measurement includes (a) converting the analog signal into a digital output and/or (b) determining pressure based at least in part on the analog signal, the digital output, and/or a relationship between measurements captured by a photosensor and pressure applied to the diaphragm.

Capturing a pressure measurement can include detecting an amount of light reflected from the diaphragm when no pressure (e.g., other than atmospheric pressure) is applied to the diaphragm or when a positive or negative pressure is applied to the diaphragm. For example, the method 860 can detect an amount of light reflected from the diaphragm when no pressure is applied to the diaphragm to determine a zero-offset value for pressure calculations. As another example, the method 860 can detect an amount of light reflected from the diaphragm when positive and/or negative test pressures are applied to the diaphragm to determine a relationship between an amount of light detected by one or more photosensors of the pressure sensor and pressures applied to the diaphragm. As still another example, the method 860 can detect an amount of light reflected from the diaphragm to measure pressure of fluid within the cavity of the portion of the disposable set.

In these and other embodiments, capturing a pressure measurement can include compensating for effects due to temperature, variations in voltage or current, and/or aging. Compensating for the effects can include detecting multiple amounts of light reflected from the diaphragm using multiple photosensors, such as a first photosensor and a second photosensor (e.g., to determine a differential signal of the amount of light reflected from the diaphragm). In these and other embodiments, compensating for the effects can include capturing one or more temperature measurements using a temperature sensor, such as one or more temperature measurements of a light source and/or a photosensor. In these and still other embodiments, compensating for the effects can include detecting, using a photosensor, a first portion of light emitted from a light source but not directed toward the diaphragm while a second portion of the light is emitted from the light source and directed toward the diaphragm. Additionally, or alternatively, compensating for the effects can include projecting light from a light source directly (e.g., without reflecting the light off of the diaphragm) onto a photosensor configured to detect a portion of light reflected from the diaphragm. Compensating for the effects can include (a) providing similar or identical photosensors and/or light sources; (b) supplying multiple photosensors and/or multiple light sources power from a same power source and/or (b) positioning photosensors and/or light sources as close to one another as is practical.

At block 864, the method 860 continues by determining whether a fluid pressure measured by the pressure sensor is within a safe operating range. In some embodiments, the safe operating range can include fluid pressures at which there is little risk of patient harm or discomfort from pressure of the fluid as it is introduced into or is drained out of the patient. For example, a safe operating range can include pressures from about -1.5 kPa to about +1.5 kPa. If the method 860 determines that the fluid pressure measured by the pressure sensor is within the safe operating range, the method 860 can return to block 863 to capture a next pressure measurement of fluid within the cavity 316 of the portion 307 of the disposable set. On the other hand, if the method 860 determines that the fluid pressure measured by the pressure sensor exceeds or is below the safe operating range, the method 860 can proceed to block 865.

At block 865, the method 860 continues by interrupting the flow of fluid through the disposable set and/or taking one or more other remedial actions. In some embodiments, interrupting the flow of fluid can include interrupting a dialysis cycle. In these and other embodiments, the one or more other remedial actions can include bringing the fluid pressure to within the safe operating range (e.g., using a pump or damping device of the system). In these and still other embodiments, the one or more other remedial actions can include generating and/or triggering an alert (e.g., to the user or patient) that pressure of the fluid is outside of the safe operating range.

Although the steps of the method 860 are discussed and illustrated in a particular order, the method 860 illustrated in FIG. 8 is not so limited. In other embodiments, the method 860 can be performed in a different order. In these and other embodiments, any of the steps of the method 860 can be performed before, during, and/or after any of the other steps of the method 860. Moreover, a person of ordinary skill in the relevant art will recognize that the illustrated method 860 can be altered and still remain within these and other embodiments of the present technology. For example, one or more steps of the method 860 illustrated in FIG. 8 can be omitted and/or repeated in some embodiments. As a specific example, the blocks 862 and 863 can be repeated several times in some embodiments. A first iteration of the blocks 862 and 863 can be performed without pressure (e.g., other than atmospheric pressure) applied to the diaphragm, for example, to determine a zero-offset value for pressure calculations. One or more subsequent iterations of the blocks 862 and 863 can be performed with one or more test pressures applied to the diaphragm to determine a relationship between an amount of light detected by one or more photosensors of the pressure sensor and pressures applied to the diaphragm. And still further subsequent iterations of the blocks 862 and 863 can be executed to measure pressure of fluid within the cavity of the portion of the disposable set. All or a subset of these iterations of the blocks 862 and 863 can be performed and/or be specific to a given diaphragm, a given pressure range, and/or a given optical geometry such that all or a subset of these iterations of the blocks 862 and 863 can be repeated for a different diaphragm, a different pressure range, and/or a different optical geometry.

Although not shown so as to avoid unnecessarily obscuring the description of the embodiments of the technology, any of the devices, systems, and methods described above can include and/or be performed by a computing device configured to direct and/or arrange components of the systems and/or to receive, arrange, store, analyze, and/or otherwise process data received, for example, from the APD system and/or other components of the APD system (e.g., from pressure sensors, one or more photosensors, temperature sensors etc.). As such, such a computing device includes the necessary hardware and corresponding computer-executable instructions to perform these tasks. More specifically, a computing device configured in accordance with an embodiment of the present technology can include a processor, a storage device, input/output device, one or more sensors, and/or any other suitable subsystems and/or components (e.g., displays, speakers, communication modules, etc.). The storage device can include a set of circuits or a network of storage components configured to retain information and provide access to the retained information. For example, the storage device can include volatile and/or non-volatile memory. As a more specific example, the storage device can include random access memory (RAM), magnetic disks or tapes, and/or flash memory.

The computing device can also include (e.g., non-transitory) computer readable media (e.g., the storage device, disk drives, and/or other storage media) including computer-executable instructions stored thereon that, when executed by the processor and/or computing device, cause the systems to perform one or more of the methods described herein. Moreover, the processor can be configured for performing or otherwise controlling steps, calculations, analysis, and any other functions associated with the methods described herein.

In some embodiments, the storage device can store one or more databases used to store data collected by the systems as well as data used to direct and/or adjust components of the systems. In one embodiment, for example, a database is an HTML file designed by the assignee of the present disclosure. In other embodiments, however, data is stored in other types of databases or data files.

One of ordinary skill in the art will understand that various components of the systems (e.g., the computing device) can be further divided into subcomponents, or that various components and functions of the systems may be combined and integrated. In addition, these components can communicate via wired and/or wireless communication, as well as by information contained in the storage media.

### C. Examples

Several aspects of the present technology are set forth in the following examples. Although several aspects of the present technology are set forth in examples specifically directed to systems, methods, and computer-readable mediums; any of these aspects of the present technology can similarly be set forth in examples directed to any of systems, devices, methods, and/or computer-readable mediums in other embodiments.

1. An automated peritoneal dialysis (APD) system, comprising:
a disposable set, wherein-
   at least a portion of the disposable set includes a diaphragm positioned over an opening in a cavity,
   the diaphragm is configured to deform in response to a force applied against the diaphragm due to pressure of fluid within the cavity, and
   the diaphragm has an outer surface and an inner surface opposite the outer surface; and
a pressure sensor configured to measure the pressure of the fluid within the cavity, the pressure sensor including a light source and a photosensor, wherein, during operation—
   the light source is configured to irradiate the outer surface of the diaphragm with light, and
   the photosensor is configured to measure an amount of the light that is reflected off of the outer surface of the diaphragm and directed toward the photosensor.

2. The APD system of example 1 wherein the disposable set further includes a reflector disposed on the outer surface of the diaphragm and configured to reflect the light.

3. The APD system of example 1 or example 2 wherein:
the pressure sensor further includes a collimating element positioned between the light source and the outer surface of the diaphragm;
the light is collimated light; and
the collimating element is configured to collimate first light emitted from the light source into the collimated light.

4. The APD system of any of examples 1-3 wherein:
the photosensor is a first photosensor;
the amount of the light is a first amount of the light;
the pressure sensor further includes a second photosensor separate from the first photosensor; and
the second photosensor is directed toward the outer surface of the diagram and is configured to measure a second amount of the light that is reflected off of the outer surface of the diaphragm and is directed toward the second photosensor.

5. The APD system of any of examples 1-4 wherein:
the photosensor is a first photosensor;
the pressure sensor further includes a second photosensor separate from the first photosensor; and
the second photosensor is configured to measure an amount of the light that is emitted from the light source and is not reflected off of the outer surface of the diaphragm.

6. The APD system of any of examples 1-5 wherein:
the light source is a first light source;
the pressure sensor further includes a second light source separate from the first light source; and
the second light source is configured to project second light onto the photosensor without the second light reflecting off of the outer surface of the diaphragm.

7. The APD system of any of examples 1-6 wherein:
the pressure sensor further includes a temperature sensor disposed on or proximate the light source; and
the temperature sensor is configured to capture one or more measurements of temperature of the light source.

8. The APD system of any of examples 1-7 wherein:
the pressure sensor further includes a temperature sensor disposed on or proximate the photosensor; and
the temperature sensor is configured to capture on or more measurements of temperature of the photosensor.

9. A method of measuring pressure of fluid within a disposable set of an automated peritoneal dialysis (APD) system, the method comprising:
irradiating an outer surface of a diaphragm with light using a light source, the diaphragm positioned over an opening to a cavity of the disposable set that is configured to contain the fluid; and
measuring, using a photosensor, an amount of the light reflected off of the outer surface of the diaphragm and directed toward the photosensor.

10. The method of example 9, further comprising aligning the outer surface of the diaphragm with the light source and the photosensor.

11. The method of example 9 or example 10 wherein:
irradiating the outer surface of the diaphragm includes irradiating the outer surface of the diaphragm in an absence of a force applied against the diaphragm due to the pressure of the fluid within the disposable set;
measuring the amount of the light includes determining a zero-offset value; and
determining the zero-offset value includes measuring the amount of light in the absence of the force applied against the diaphragm due to the pressure of the fluid within the disposable set.

12. The method of any of examples 9-11 further comprising determining a relationship between amounts of light measured by the photosensor and deformation of the diaphragm in response to forces applied against the diaphragm due to pressures of the fluid within the disposable set.

13. The method of any of examples 9-12 wherein:
irradiating the outer surface of the diaphragm includes irradiating the outer surface of the diaphragm in a presence of a force applied against the diaphragm due to the pressure of the fluid within the disposable set; and
measuring the amount of the light includes measuring the amount of light in the presence of the force applied against the diaphragm due to the pressure of the fluid within the disposable set.

14. The method of any of examples 9-13, further comprising determining the pressure of the fluid within the disposable set based at least in part on the amount of the light reflected off of the outer surface of the diaphragm and directed toward the photosensor.

15. The method of example 14, further comprising comparing the pressure of the fluid to a safe operating pressure range.

16. The method of example 15, further comprising interrupting flow of the fluid through the disposable set when the pressure of the fluid is outside of the safe operating pressure range.

17. The method of any of examples 9-16, further comprising capturing one or more temperature measurements of the light source or the photosensor.

18. The method of any of examples 9-17 wherein:
the photosensor is a first photosensor; and
the method further comprises measuring, using a second photosensor separate from the first photosensor, an amount of the light that is emitted from the light source and is not reflected off of the outer surface of the diaphragm.

19. The method of any of examples 9-18 wherein:
the light source is a first light source;
the light is first light; and
the method further comprises projecting second light onto the photosensor without reflecting the second light off of the outer surface of the diaphragm.

20. The method of any of examples 9-19 wherein:
the photosensor is a first photosensor;
the amount of the light is a first amount of the light; and
the method further comprises measuring, using a second photosensor separate from the first photosensor, a second amount of the light reflected off of the outer surface of the diaphragm and directed toward the second photosensor.

21. The method of any of examples 9-20 wherein:
the light is collimated light; and
irradiating the outer surface of the diaphragm with collimated light includes collimating first light emitted from the light source into the collimated light.

22. The method of any of examples 9-21 wherein measuring the amount of the light includes compensating for effects due to temperature, aging, variations in voltage supplied to the photosensor, or variations in current supplied to the light source.

23. A non-transitory, computer-readable medium having instructions stored thereon that, when executed by one or more processors of an automated peritoneal dialysis (APD) system, cause the APD system to perform a method comprising:
irradiating an outer surface of a diaphragm of a disposable set with light using a light source, the diaphragm positioned over an opening to a cavity of the disposable set that is configured to contain fluid; and
measuring, using a photosensor, an amount of the light reflected off of the outer surface of the diaphragm and directed toward the photosensor.

### C. Conclusion

From the foregoing, it will be appreciated that specific embodiments of the technology have been described herein for purposes of illustration, but well-known structures and functions have not been shown or described in detail to avoid unnecessarily obscuring the description of the embodiments of the technology. To the extent any materials incorporated herein by reference conflict with the present disclosure, the present disclosure controls. Where the context permits, singular or plural terms can also include the plural or singular term, respectively. Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. As used herein, the phrase “and/or” as in “A and/or B” refers to A alone, B alone, and both A and B. Where the context permits, singular or plural terms can also include the plural or singular term, respectively. Additionally, the terms “comprising,” “including,” “having” and “with” are used throughout to mean including at least the recited feature(s) such that any greater number of the same feature and/or additional types of other features are not precluded.

Furthermore, as used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. Moreover, the terms “connect” and “couple” are used interchangeably herein and refer to both direct and indirect connections or couplings. For example, where the context permits, element A “connected” or “coupled” to element B can refer (i) to A directly “connected” or directly “coupled” to B and/or (ii) to A indirectly “connected” or indirectly “coupled” to B.

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, while steps are presented in a given order, alternative embodiments can perform steps in a different order. As another example, various components of the technology can be further divided into subcomponents, and/or various components and/or functions of the technology can be combined and/or integrated. Furthermore, although advantages associated with certain embodiments of the technology have been described in the context of those embodiments, other embodiments can also exhibit such advantages, and not all embodiments need necessarily exhibit such advantages to fall within the scope of the technology.

It should also be noted that other embodiments in addition to those disclosed herein are within the scope of the present technology. For example, embodiments of the present technology can have different configurations, components, and/or procedures in addition to those shown or described herein. Moreover, a person of ordinary skill in the art will understand that these and other embodiments can be without several of the configurations, components, and/or procedures shown or described herein without deviating from the present technology. Accordingly, the disclosure and associated technology can encompass other embodiments not expressly shown or described herein.

What is claimed is:

1. An automated peritoneal dialysis (APD) system, comprising:
a disposable set,
wherein at least a portion of the disposable set includes a diaphragm positioned over an opening in a cavity, the diaphragm is configured to deform in response to a force applied against the diaphragm due to pressure of fluid within the cavity, and the diaphragm has an outer surface and an inner surface opposite the outer surface; and
a pressure sensor configured to measure the pressure of the fluid within the cavity, the pressure sensor including a light source and a photosensor, and
wherein, during operation the light source is configured to irradiate the outer surface of the diaphragm with light, and the photosensor is configured to measure an amount of the light that is reflected off of the outer surface of the diaphragm and directed toward the photosensor.

2. The APD system of claim 1, wherein the disposable set further includes a reflector disposed on the outer surface of the diaphragm and configured to reflect the light.

3. The APD system of claim 1, wherein:
the pressure sensor further includes a collimating element positioned between the light source and the outer surface of the diaphragm;
the light is collimated light; and
the collimating element is configured to collimate first light emitted from the light source into the collimated light.

4. The APD system of claim 1, wherein:
the photosensor is a first photosensor;
the amount of the light is a first amount of the light;
the pressure sensor further includes a second photosensor separate from the first photosensor; and
the second photosensor is directed toward the outer surface of the diagram and is configured to measure a second amount of the light that is reflected off of the outer surface of the diaphragm and is directed toward the second photosensor.

5. The APD system of claim 1, wherein:
the photosensor is a first photosensor;
the pressure sensor further includes a second photosensor separate from the first photosensor; and
the second photosensor is configured to measure an amount of the light that is emitted from the light source and is not reflected off of the outer surface of the diaphragm.

6. The APD system of claim 1, wherein:
the light source is a first light source;
the pressure sensor further includes a second light source separate from the first light source; and
the second light source is configured to project second light onto the photosensor without the second light reflecting off of the outer surface of the diaphragm.

7. The APD system of claim 1, wherein:
the pressure sensor further includes a temperature sensor disposed on or proximate the light source; and
the temperature sensor is configured to capture one or more measurements of temperature of the light source.

8. The APD system of claim 1, wherein:
the pressure sensor further includes a temperature sensor disposed on or proximate the photosensor; and
the temperature sensor is configured to capture on or more measurements of temperature of the photosensor.

9. A method of measuring pressure of fluid within a disposable set of an automated peritoneal dialysis (APD) system, the method comprising:
irradiating an outer surface of a diaphragm with light using a light source, the diaphragm positioned over an opening to a cavity of the disposable set that is configured to contain the fluid; and
measuring, using a photosensor, an amount of the light reflected off of the outer surface of the diaphragm and directed toward the photosensor.

10. The method of claim 9, further comprising aligning the outer surface of the diaphragm with the light source and the photosensor.

11. The method of claim 9, wherein:
irradiating the outer surface of the diaphragm includes irradiating the outer surface of the diaphragm in an absence of a force applied against the diaphragm due to the pressure of the fluid within the disposable set;
measuring the amount of the light includes determining a zero-offset value; and
determining the zero-offset value includes measuring the amount of light in the absence of the force applied against the diaphragm due to the pressure of the fluid within the disposable set.

12. The method of claim 9, further comprising determining a relationship between amounts of light measured by the photosensor and deformation of the diaphragm in response to forces applied against the diaphragm due to pressures of the fluid within the disposable set.

13. The method of claim 9, wherein:
irradiating the outer surface of the diaphragm includes irradiating the outer surface of the diaphragm in a presence of a force applied against the diaphragm due to the pressure of the fluid within the disposable set; and
measuring the amount of the light includes measuring the amount of light in the presence of the force applied against the diaphragm due to the pressure of the fluid within the disposable set.

14. The method of claim 9, further comprising determining the pressure of the fluid within the disposable set based at least in part on the amount of the light reflected off of the outer surface of the diaphragm and directed toward the photosensor.

15. The method of claim 14, further comprising comparing the pressure of the fluid to a safe operating pressure range.

16. The method of claim 15, further comprising interrupting flow of the fluid through the disposable set when the pressure of the fluid is outside of the safe operating pressure range.

17. The method of claim 9, further comprising capturing one or more temperature measurements of the light source or the photosensor.

18. The method of claim 9, wherein:
the photosensor is a first photosensor; and
the method further comprises measuring, using a second photosensor separate from the first photosensor, an amount of the light that is emitted from the light source and is not reflected off of the outer surface of the diaphragm.

19. The method of claim 9, wherein:
the light source is a first light source; the light is first light; and the method further comprises projecting second light onto the photosensor without reflecting the second light off of the outer surface of the diaphragm.

20. The method of claim 9, wherein:
the photosensor is a first photosensor;
the amount of the light is a first amount of the light; and
the method further comprises measuring, using a second photosensor separate from the first photosensor, a second amount of the light reflected off of the outer surface of the diaphragm and directed toward the second photosensor.

21. The method of claim 9, wherein:
the light is collimated light; and
irradiating the outer surface of the diaphragm with collimated light includes collimating first light emitted from the light source into the collimated light.

22. The method of claim 9, wherein measuring the amount of the light includes compensating for effects due to temperature, aging, variations in voltage supplied to the photosensor, or variations in current supplied to the light source.

23. A non-transitory, computer-readable medium having instructions stored thereon that, when executed by one or more processors of an automated peritoneal dialysis (APD) system, cause the APD system to perform a method comprising:
irradiating an outer surface of a diaphragm of a disposable set with light using a light source, the diaphragm positioned over an opening to a cavity of the disposable set that is configured to contain fluid; and
measuring, using a photosensor, an amount of the light reflected off of the outer surface of the diaphragm and directed toward the photosensor.
